SEQUENCE LISTING (6 sequences)

SEQ ID NO: 1 - DNA, 531 bp, Methanothermobacter thermoautotrophicus
gtgatcaatatgagggaaaagttaatgggagtaatcccgcttatggttgcccttgtgtttgtggtggcaataggtgcatacagctcaccatcctacgcggcaacagcaagccagacagttacagttacagtgccagaggccatctcaatagttgtaccgaatgttaacttcgggagcattgccgcaggaagtgaaggaacaagccctgctttcacagtaagtaacacaggtaacgtcaagatagacctctacgtcaaggcagacgcatcagcattcacaagcccaactgctacagatacaataccaataacagggttcaatattttcagtaatgtcacaggtaactaccagaacatcacaaccagctcccttaagatatatgacaacatgaacaaggcatcccagggagcaggtaccccgacaacatggacaacaacactcagactctttgtacccccattcacagaggacggcacatacacaataacaaacacatacacagcagtgaagcataactcacctgcaccataa

SEQ ID NO: 2 - protein, 176 aa, Methanothermobacter thermoautotrophicus
MINMREKLMGVIPLMVALVFVVAIGAYSSPSYAATASQTVTVTVPEAISIVVPNVNFGSIAAGSEGTSPAFTVSNTGNVKIDLYVKADASAFTSPTATDTIPITGFNIFSNVTGNYQNITTSSLKIYDNMNKASQGAGTPTTWTTTLRLFVPPFTEDGTYTITNTYTAVKHNSPAP

SEQ ID NO: 3 - DNA, 513 bp, Methanothermobacter thermoautotrophicus
  MISC_FEATURE: Isolate DeltaH
atgtacagccacgaatggatgggtgtgctcttcatcctgctcctccttccggtgccatttgcaaccatgaatacggtgcaggaagttaccgttacggtacccgagagtgttgaaataatggtcctctggcagggaagggaaaccgggaactccttcacactcacagccacagtggaacccggaaaagaatactactggccaggaggaccccagggactccagataaaggacctatccaacgtacccatcgacctctacataagagccgaaggagacctccagggcccagaaaccatacccatacagaacctcaaatacgccaactacggcatcggactccccgaaacaccactaacaacaacctacacaccggtaagaaagaactgggcagcgaaacgggatatcgatgcggtggtgcctgttgatctcagtctcacggtgccaccattcacagaacccggcgaatacagggtgagggtctaccatatagccataaggtcaccgggtacctga

SEQ ID NO: 4 - protein, 170 aa, Methanothermobacter thermoautotrophicus
  MISC_FEATURE: Isolate DeltaH
MYSHEWMGVLFILLLLPVPFATMNTVQEVTVTVPESVEIMVLWQGRETGNSFTLTATVEPGKEYYWPGGPQGLQIKDLSNVPIDLYIRAEGDLQGPETIPIQNLKYANYGIGLPETPLTTTYTPVRKNWAAKRDIDAVVPVDLSLTVPPFTEPGEYRVRVYHIAIRSPGT

SEQ ID NO: 5 - DNA, 510 bp, Methanothermobacter thermoautotrophicus
  MISC_FEATURE: Isolate DeltaH
atgcttaaaactgctggaatggtcactgcagtcatcctccttctgcttttaaaacctgcagcaggagccaccgcggtgcaggaggttaccgttacggtgcccgagagtgttgaaataatggtcctctggcagggaagggaaaccgggaactccttcacactcacagccacagtggaacccggaaaagaatactactggccaggaggaccccagggactccagataaaggacctatccaacgtacccatcgacctctacataagagccgaaggagacctccagggcccagaaaccatacccatacagaacctcaaatacgccaactacggcatcggactccccgaaacaccactaacaacaacctacacaccggtaagaaagaactggatggttaaatcagaggatgaatccctgataccggttgacctccacctcacggtaccaccagcaaccgctgccggtgtctactcagttaacatataccacatagcggtcccccatggagaatag

SEQ ID NO: 6 - protein, 169 aa, Methanothermobacter thermoautotrophicus
  MISC_FEATURE: Isolate DeltaH
MLKTAGMVTAVILLLLLKPAAGATAVQEVTVTVPESVEIMVLWQGRETGNSFTLTATVEPGKEYYWPGGPQGLQIKDLSNVPIDLYIRAEGDLQGPETIPIQNLKYANYGIGLPETPLTTTYTPVRKNWMVKSEDESLIPVDLHLTVPPATAAGVYSVNIYHIAVPHGE

(19) United States
(12) Patent Application Publication
(10) Pub. No.: US 2010/0022758 A1
(43) Pub. Date: Jan. 28, 2010

(54) PYROGLUE

(75) Inventors: Reinhard Wirth, Regensburg (DE); Christine Thoma, Landshut (DE)

Correspondence Address: FULBRIGHT & JAWORSKI L.L.P., 600 CONGRESS AVE., SUITE 2400, AUSTIN, TX, 78701, US

(73) Assignee: UNIVERSITAT REGENSBURG, Regensburg (DE)

(21) Appl. No.: 12/090,828

(22) PCT Filed: Nov. 9, 2006
(86) PCT No.: PCT/EP2006/010756

§ 371 Date: Sep. 23, 2009

(30) Foreign Application Priority Data
Nov. 11, 2005 (EP) 05024658.6

(51) Int. Cl. C07K 14/00

(52) U.S. Cl. 530/402; 530/350

(57) ABSTRACT

The present invention relates to an adhesive material being composed and/or consisting of at least one protein obtained or obtainable from fimbriae from archaea. Furthermore, the present invention relates to the use of at least one protein obtained from fimbriae from archaea for the preparation of an adhesive material and a method for the preparation of an adhesive material comprising the step of isolating and/or purifying at least one protein obtained from fimbriae from archaea.

The present invention relates to an adhesive material being composed and/or consisting of at least one protein obtained or obtainable from fimbriae from archaea. Furthermore, the present invention relates to the use of at least one protein obtained from fimbriae from archaea for the preparation of an adhesive material and a method for the preparation of an adhesive material comprising the step of isolating and/or purifying at least one protein obtained from fimbriae from archaea.

Surface organelles of prokaryotes may be differentiated into those used for motility (named flagella) and those used for adhesion (in most cases named fimbriae). In the case of eubacteria some fimbriae have been defined (at least for some species like the Enterobacteria Escherichia coli and Salmonella typhimurium) to a very high resolution, which is true for molecular and functional aspects. In the case of archaea (=archaebacteria) fimbriae remain more or less undefined, especially with respect to their function. This latter statement is supported by the fact that e.g. a GOOGLE search results in >200,000 hits for “fimbriae”, in 830 hits for ‘archaea and fimbriae’, but 0 hits for “archaeal fimbriae”. A careful check of the ‘archaea and fimbriae’ hits reveals that only very few of those actually deal with fimbriae from archaea. The nomenclature of cell surface organelles used for adhesion only can be described to be messy—see e.g. Low (1996; in Escherichia coli and Salmonella:146-157; ASM press); in most cases both the words fimbriae and pili are used synonymous. However, so far no data as to a possible function of fimbriae from archaea have been published.

Compounds of nature have since decades attracted the interest of researchers. Researchers have learned and still learn from biology how to apply attainments from nature. In particular, nanobiotechnology is an emerging area of scientific and technological opportunity. Nanobiotechnology applies, for example, compounds/structures of nature if, e.g. synthetically produced products cannot comply with extreme requirements, e.g. heat, moisture etc. One example for synthetically produced products are glues which are mainly produced by chemical synthesis. These glues, when applied to biological systems show disadvantages insofar as they leach, may be toxic and/or are incompatible with the biological system etc.

The problem underlying the present invention is the development of a glue which is heat stable and/or which can also be applied in wet and moist environments. Presently used glues are often epoxy based, cement based or based on synthetic polymers. Both the epoxy compounds and the synthetic polymers may leach and constitute a risk to the environment. Their application often requires mechanical working or kneading of the glue or sealing agent, in order to remove the water present on the surfaces. There is a need for new glues, better adapted for use in warm and/or moist environments or for underwater use and more environmentally friendly than the present products.

Furthermore, there is a need for the provision of materials and compositions which may be efficiently employed as “glue” in (nano)technology applications, like the preparation of chips, in particular DNA chips/arrays or protein chips/arrays, like antibody arrays.

The solution to said technical problem is achieved by the embodiments provided herein and as characterized in the claims. Accordingly, the present invention relates to an adhesive material being composed and/or consisting of at least one protein obtained or obtainable from fimbriae from archaea. The domain archaea (=archaebacteria) comprises according to the Systema Naturae 2000 (http://sn2000.taxonomy.nl) the phyla Crenarchaeota, Euryarchaeota and Nanoarchaeota. These phyla include further classes which are known to the skilled person. Among the phyla are, for example, halophiles or thermophiles. By using classical systematics, for example, by reference to the pertinent descriptions in “Bergey's Manual of Systematic Bacteriology” (Williams & Wilkins Co., 1984), the skilled person can determine whether a prokaryote is an archaeum. Alternatively, the affiliation of a prokaryote to the archaea can be characterized with regard to ribosomal RNA in a so called Riboprinter®. More preferably, the affiliation of a prokaryote to the archaea is demonstrated by comparing the nucleotide sequence of the 16S ribosomal RNA of such a prokaryote, or of its genomic DNA which codes for the 16S ribosomal RNA, with those of other known archaea. Another alternative for determining the affiliation of a prokaryote to the archaea is the use of species- or domain-specific PCR primers that target the 16S-23S rRNA spacer region.

In accordance with the present invention, it was surprisingly found that specific surface organelles of archaea, in particular of (hyper)thermophilic archaea, which are not used for motility of said archaea contribute significantly to the adhesion on solid surfaces, in particular to metal surfaces such as copper and/or to plastic surfaces such as PVC, PTFE, PC or nylon and the like and/or to silica surfaces and the like; see Table 3. It is also believed that said specific surface organelles of archaea contribute significantly to the adhesion on quartz surfaces and the like.

Accordingly, the present study relates in particular to surface organelles and in particular to isolated proteins/proteinaceous structures of archaea, preferably (hyper)thermophilic archaea, especially of M. thermoautotrophicus. Of course, also archaea being (extreme) halophiles, alkalophiles, or acidophiles might be the source of those proteins/structures.

The archaeum M. thermoautotrophicus ΔH (sometimes also referred to herein as “Delta H”) originally was isolated from an anaerobic sewage-sludge digester—Zeikus (1972; J. Bacteriol. 109:707-713) and later reported to possess fimbriae—Doddema (1979; FEMS Microbiol. Lett. 5:135-138). M. thermoautotrophicus ΔH was deposited under DSMZ1053.

Data as to a possible function of those fimbriae have not been published up to today. However, the analyses presented herein show that these surface organelles enable M. thermoautotrophicus to adhere onto surfaces. Other isolates of M. thermoautotrophicus (like strain Ag5) and closely related species have been found to be abundant in hydrothermal systems like e.g. Agnano-Therme (Italy). In theses habitats the cells face the problem that a constant flow of liquid would remove them from locations not possessing their optimal growth temperatures (ca. 65° C.). The cells have been described to be not motile, but to possess fimbriae. In the present application it was retested if M. thermoautotrophicus might be motile by using the fimbriae for a so-called twitching motility (as described for type IV pili of e.g. E. coli), but no indication for this could be found. Such studies were carried out in a thermomicroscope allowing studies of potential swimming behaviour up to 95° C. under strictly anaerobic conditions. On the other hand the present application shows that M. thermoautotrophicus adhered to (carbon coated) gold grids used for electron microscopy. Especially it was observed that 100% of all cells adhering to the gold grids did possess a multitude of fimbriae, whilst only some 50% of cells from the liquid growth medium were fimbriated. Therefore these surface appendages are believed to function as adhesins.

Since most other archaea face the same problem like M. thermoautotrophicus, namely to steadily stay in very sharply defined regions in their natural habitat (otherwise they would be swamped away form places having e.g. temperatures they need for growth) the present invention also provides for the fact that other fimbrial proteins of archaea have a similar adhesion function. The advantage of using adhesins of archaea over those from eubacteria as “molecular glue” lies in the fact that in many cases these proteins are optimised for extreme conditions—in the case of M. thermoautotrophicus e.g. to temperatures between 0-70° C., 10-70° C., 20-70° C., 30-70° C., 40-70° C., 50-70° C., 60-70° C., or around or above 70° C.

The applications for such a protein glue are seen in the field of nano(bio)technology. Proteins acting as molecular cement to connect part A to part B do not have the disadvantage of chemicals which might interfere with the biological functions of one of the parts. Quantum dots e.g. have to be functionalised by a shell of polyacrylic acid to allow conjugation to macromolecules and ligands.

Archaeal Fbr (fimbrial) proteins—optimised e.g. for low pH (e.g. Sulfolobus), high salt (e.g. Halobacterium), high pH (e.g. Natrialba), high temperature (many hyperthermophilic archaea, like Thermococcus or Pyrococcus)—are attractive adhesive materials to be employed in a variety of uses, like in medical settings, as well as in technologies like the use in the preparation of protein chips or nucleic acid molecule chips. Also the use in nanotechnology is envisaged.

Halophilic archaea are divided into slightly halophilic having optimal growth at 1-5% (w/v)NaCl, moderately halophilic with optimal growth at 5-18% (w/v)NaCl and extremely halophilic with optimal growth above 18% (w/v) NaCl. Also in the present context, halotolerant archaea are defined as microorganisms selected from the following types: slightly halotolerant which grow at NaCl concentrations up to 6-8% (w/v) NaCl, moderately halotolerant which grow at NaCl concentrations up to 18-20% (w/v) NaCl, and extremely halotolerant growing at NaCl concentrations up to and above 20% (w/v) and occasionally to the point of saturation of NaCl (approx. 36% (w/v) NaCl).

Acidophilic archaea can be divided into moderate acidophilic archaea growing above pH 4, acidophilic archaea growing between pH 1.5 to 4 and extreme acidophilic archaea growing between pH 0 to 2. Alkaliphilic archaea can be divided into moderate alkaliphilic ones growing up to pH 9, into alkaliphilic ones growing best at pH 8.5 to 10; extreme alkaliphilic archaea do possess ph optima for growth of 11 or even higher.

Since it is shown herein that the fimbrin from M. thermoautotrophicus is a glycoprotein the question arises how one can obtain sufficient amounts for (nano)technological as well as medical applications.

A first alternative might be the isolation of the material directly from cells after growth. For example, Example 1 demonstrates a direct isolation procedure of material from cells after growth.

In another example for direct isolation of material, the skilled person can try to directly obtain fimbriae from supernatant of grown cultures of archaea having fimbriae, for example, from M. thermoautotrophicus. Namely, cells can be removed from culture medium by centrifugation, for example, at 16,000×g and the supernatant can be mixed with polyethylene glycol (PEG), e.g., PEG 2000, 4000 or preferably PEG 6000 which is available from various suppliers known in the art. The supernatant can be adjusted to 20, 19, 18, 17, 16, 15, 14, 13, 12, 11, 10, 9, 8, 7, 6, 5 or less percent (%), preferably to 10.5% final concentration with polyethylene glycol, preferably with PEG 6000 plus 10, 9, 8, 7, 6, 5, 4, 3, 2, 1 or preferably 5.8% NaCl. After incubation for at least 24, 18 or preferably 12 hours at a temperature between 10° C. and 4° C., preferably at 4° C., the material is centrifuged at, for example, 11,000×g for at least 30 min; the resulting pellet could be further purified, for example, via CsCl centrifugation with the same procedure as outlined in Example 1 hereinbelow. First data show that this procedure results in substantial higher yields than shearing fimbriae from cells concentrated via prior centrifugation; see also Example 4 hereinbelow.

A second alternative is the use of eukaryotic cells—like CHO cells or insect cells—and expression vectors developed for them for production of recombinant proteins. Potential insect systems would be e.g. the DES-system (Drosophila expression system by Invitrogen) or the Sf9/Sf21 system (ovarian cells from the butterfly Spodoptera frugiperda). An attractive alternative might be the use of the yeast Kluyveromyces lactis—in case of the K. lactis protein expression system (of NEB) the glycosylated proteins would be secreted into the growth medium.

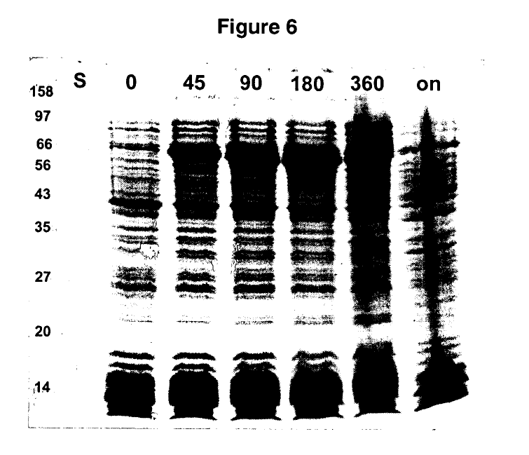

A further alternative is the expression of archaeal fimbrins in bacteria, especially in Escherichia coli. It has to be noted that the IMPACT-system (by NEB) allowed the expression of a fusion protein of expected size. This fusion protein was expressed from pTYB2 (using the unique NdeI and SmaI restriction sites) and is obtained as Fimbrin—Intein—Chitin-binding-domain (see FIG. 6). A protein of the expected size for M. thermoautotrophicus fimbrin may be obtained after induction of expression via IPTG addition to the recombinant E. coli strain ER2566, lysis of cells, binding of the fusion protein onto chitin beads, washing of this affinity column, induction of intein-splicing by addition of DTT, and finally elution of the fimbrin (the resulting fusion protein of Intein—Chitin-binding-domain remains bound to the column).

Another alternative is the use of a yeast expression system as described in WO 02/00879. In particular said PCT-application describes host cells derived from unicellular or filamentous fungi which are lacking a key enzyme of yeast glycosylation. Accordingly, said host cells are not capable of glycosylating proteins in a yeast-like manner leading to high-mannose structures. Thus, after transforming said modified host cells with enzymes involved in glycosylation processes in archaea, it could be envisaged that a desired archaeal protein is produced by yeast having a glycosylation pattern as occurring in its natural host. Very recently Voisin (2005; J. Biol. Chem. 280:16586-16593) was able to determine the glycosylation pattern of Methanococcus voltae flagellins using microtechniques. Accordingly, it is expected that glycosylating enzymes of archaea can be identified and isolated and, thus, can be used for the aforementioned purpose when expressing an archaeal protein in an artificial yeast expression system.

A further alternative that could be envisaged is to express the genes directly in archaea.

Fbr proteins (fimbrins) purified from sheared fimbrins or expressed in recombinant form may be applied onto various surfaces, like e.g. metals, silicon, quartz, alumina, silica, polymers, etc. A test system for the “adhesive capacity” of a given archaeal fimbrin is provided as follows: After a certain binding time to surfaces, these are washed thoroughly and tested for adherence of the Fbr proteins. Detection of bound Fbr proteins might be via immunological or by direct staining methods. In the first case antibodies against purified Fbr proteins are applied onto the surfaces and after a certain binding time unbound antibodies are removed by washing steps. Antibodies bound to Fbr proteins which themselves adhere to the surfaces can be detected via various available techniques including secondary antibodies coupled to enzymes resulting in colour development, resulting in chemiluminescence, etc. In the second case one might label bound Fbr proteins with fluorescent dyes like e.g. AlexaFluor succinimidyl esters. The present inventors have been successful to stain fimbriae of M. thermoautotrophicus under in vivo conditions using AlexaFluor succinimidyl esters.

The person skilled in the art can easily obtain archaeal fimbrins by methods known in the art and by methods provided herein. In accordance with this invention, the term “fimbrin” is synonymous with the term “Fbr proteins”. Notably, the Fbr proteins described herein are different from Fla proteins of flagella.

In principle flagella and fimbriae differ in the following aspects:

| Flagella | Fimbriae |
|---|---|
| Used for swimming by rotation of the | Used for adhesion; no active motion |
| organelle. | reported. |
| Bacterial flagella have a diameter of ca. | Bacterial proteinaceous fimbriae have a |
| 20 nm with a central channel of ca. 2 nm. | diameter of 2-8 nm and differ in their |
| In most cases bacterial flagellin subunits | length. Fimbrin subunits range in size |
| are not glycosylated. | from 14 to 30 kDa. |
| Archaeal flagella have a diameter of ca. | Archaeal fimbriae up to now are defined |
| 10-15 nm without a central channel. In | only by possessing clearly a smaller |
| most cases archaeal flagellin subunits are | diameter than archaeal flagella; e.g. in |
| glycosylated. | the range of 5 nm. |

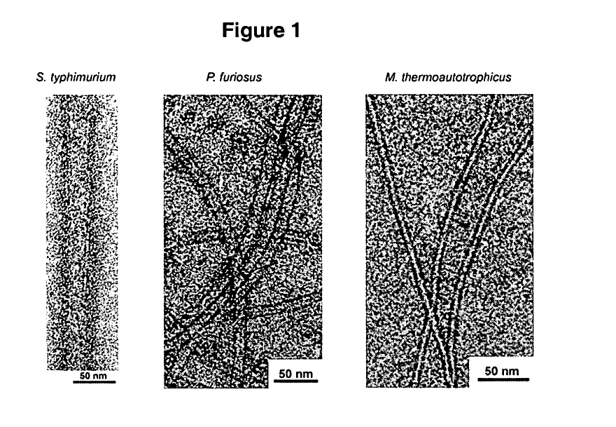

The statements given above in principle define archaeal fimbriae as relatively thin cell surface organelles of archaea for which no function as a flagellum has been shown. In FIG. 1 a comparison of bacterial flagella, archaeal flagella, and fimbriae from M. thermoautotrophicus—especially with respect to their diameter—is given.

As mentioned above, no functional studies for archaeal fimbriae have been published. The data presented herein however show that M. thermoautotrophicus is able to adhere onto surfaces via its fimbriae. Since the present invention identifies for the first time the protein constituting such a surface organelle it is proposed to use it as a new kind of “pyroglue”. In the following the new archaeal fimbrial subunit protein will be called Fbr or fimbrin.

As is evident from the appended experimental part of this invention, the “fimbrin” to be employed in context of this invention relates to proteins derived from the non-membrane associated part of the “fimbriae” of archaea. Said fimbrins are proteins constituting the long, thin filaments of archaea. Fimbrins may be glycosylated. Accordingly, for example the fimbrins to be employed in context of this invention relate, inter alia, to the fimbrin proteins “hypothetical protein Mth60”, “hypothetical protein Mth383” and “hypothetical protein Mth382” from Methanothermobacter thermoautotrophicus. Other related archaeal proteins might be hypothetical proteins MA2392 (Methanosarcina mazei), rrnAC3056 (Haloarcula maris mortui), MMP0236 (Methanococcus maripaludis), etc. as listed in Table 1. Potentially related proteins from bacteria are listed in Table 2.

Corresponding amino acid sequences are illustratively given in the appendix as “fimbrin sequences from archaea”. The present invention also envisages that the fimbrin is encoded by a nucleic acid molecule encoding the archaeal proteins as listed in Table 1. It is also believed that one or more of the proteins as listed in Table 2 may contain an amino acid sequence which could function as a fimbrin.

TABLE 1
Comparison of fimbrin Mth60 against sequenced archaeal genomes (BLAST)

| | | Accession number | |
|---|---|---|---|
| # | Protein characterization (species) | (Entrez protein) | E-value |
| 1 | Hypothetical protein MTH60 | NP_275203.1 | 5e−80 |
| | (Methanothermobacter thermoautotrophicus | | |
| | str. Delta H) | | |
| 2 | Hypothetical protein MTH383 | NP_275526.1 | 0.074 |
| | (Methanothermobacter thermoautotrophicus | | |
| | str. Delta H) | | |
| 3 | Hypothetical protein MTH382 | NP_275525.1 | 0.096 |
| | (Methanothermobacter thermoautotrophicus | | |
| | str. Delta H) | | |
| 4 | Hypothetical protein MA2392 | NP_617298.1 | 0.62 |
| | (Methanosarcina acetivorans C2A) | | |
| 5 | Hypothetical protein rrnAC3056 | YP_137481.1 | 1.1 |
| | (Haloarcula marismortui ATCC 43049) | | |
| 6 | Hypothetical protein MMP0236 | NP_987356.1 | 1.4 |
| | (Methanococcus maripaludis S2) | | |
| 7 | Cell surface protein | NP_619165.1 | 1.8 |
| | (Methanosarcina acetivorans C2A) | | |
| 8 | Amylopullulanase related protein | NP_393607.1 | 2.4 |
| | (Thermoplasma acidophilum DSM 1728) | | |
| 9 | COG5651: PPE-repeat proteins | ZP_00297222.1 | 2.4 |
| 10 | COG1520: FOG: WD40-like repeat | ZP_00294634.1 | 3.1 |

TABLE 2
Comparison of fimbrin Mth60 against sequenced bacterial genomes (BLAST)

| | | Accession number | |
|---|---|---|---|
| # | Protein characterization (species) | (Entrez protein) | E-value |
| 1 | Hypothetical protein L107870 | NP_268013.1 | 0.17 |
| | (Lactococcus lactis subsp. lactis I11403) | | |
| 2 | COG1049: Aconitase B | ZP_00339033.1 | 1.1 |
| 3 | Putative hemolysin | YP_071037.1 | 1.1 |
| | (Yersinia pseudotuberculosis IP 32953) | | |
| 4 | Putative hemolysin (Yersinia pestis biovar | NP_993634.1 | 1.1 |
| | Medievalis str. 91001) | | |
| 5 | Putative hemolysin (Yersinia pestis CO92) | NP_406024.1 | 1.1 |
| 6 | Putative hemagglutinin-like secreted protein | NP_669014.1 | 1.1 |
| | (Yersinia pestis KIM) | | |
| 7 | Cell wall surface anchor family protein | NP_967691.1 | 1.1 |
| | (Bdellovibrio bacteriovorus HD100) | | |
| 8 | COG5295: Autotransporter adhesin | ZP_00242706.1 | 1.1 |
| | (Rubrivivax gelatinosus PM1) | | |
| 9 | Hypothetical protein SYNW0953 | NP_897046.1 | 1.8 |
| | (Synechococcus sp. WH 8102) | | |
| 10 | Collagen triple helix repeat protein | NP_832177.1 | 2.4 |
| | (Bacillus cereus ATCC 14579) | | |
| 11 | Putative surface-exposed virulence protein | NP_462381.1 | 2.4 |
| | (Salmonella typhimurium LT2) | | |
| 12 | Putative hemagglutinin/hemolysin related | NP_967218.1 | 3.1 |
| | protein (Bdellovibirio bacteriovorus HD100) | | |

Thus, archaeal fimbrins comprise, but are not limited to the archaeal fimbrin shown in SEQ ID NOs: 2, 4, 6 or shown in Table 1, or as encoded by nucleic acid molecules as shown in any one of SEQ ID NOs: 1, 3, 5 or nucleic acid molecules encoding the archaeal proteins as listed in Table 1. The proteins listed in Table 2 may comprise an amino acid sequence which could function as a fimbrin. Thus, the proteins listed in Table 2 and the nucleic acid molecules encoding them are also envisaged.

A particular preferred fimbrin in context of this invention is the single fimbrin obtainable from M. thermoautotrophicus, in particular from M. thermoautotrophicus strain AG5 (Bakterienbank Regensburg) or strain ΔH as deposited under DSMZ1053. The corresponding Fbr proteins/fimbrins are of particular use in the preparation of the adhesive material(s)/glue(s) as disclosed herein.

The present inventors now make available a characterised and purified fimbrin with many uses in medicine and other technical applications as disclosed in the following description, examples and claims.

The present invention makes available a substantially pure adhesive protein, namely archaeal fimbrin, comprising preferably, but not limited to, the amino acid sequences as shown in anyone of SEQ ID Nos. 2, 4, 6 (or fragments thereof) or archaeal proteins as listed in Table 1 (or fragments thereof), including functionally equivalent fragments or variants thereof. As mentioned above, the proteins listed in Table 2 may contain an amino acid sequence which could function as a fimbrin.

The adhesive property of the archaeal fimbrins, in particular the fimbrin obtainable form M. thermoautotrophicus, as described herein is particularly useful in medical as well as in technological settings.

It is of note that in the uses provided herein, it is also envisaged that a mixture of fimbrin proteins (e.g. a mixture of adhesive fimbrins derived and/or obtainable from different species) be employed. Accordingly, the term “at least one fimbrin” as used herein also means that mixtures of adhesive fimbrins (derived from archaeal fimbriae) be used in the preparation of the inventive adhesive material/glue.

For example, the adhesive fimbrin may be used in medical applications, for example as a component in wound dressings and bandages, in particular in such applications where the biodegradable properties of the protein are needed. It is also envisaged that the adhesive property of archaeal fimbrins be employed in the coating of (medical) bands and strings. Since the archaeal fimbrins as documented herein have an ability to attach to surfaces, and to form an attachment between surfaces, they may be used as a tissue adhesive. The adhesive capability of fimbrin (Fbr protein) may, accordingly, be used as an adhesive for plasters, adhesives, bandages, patches and dressings etc. The protein may also be useful in orthopaedics as a glue to keep or hold joint replacements together. It is also envisaged to use the adhesive properties of the fimbrins as surface coating of medical and/or surgical devices and tools, e.g. stents, chirurgical nails, or transplants. The use of the adhesive fimbrins derived from archaeal fimbrin in dental medicine is also envisaged, for example in the anchorage/attachment of artificial tooth parts or crowns. Furthermore, the use of the adhesive fimbrins as provided herein in dental restoration or for dental implants is envisaged.

One embodiment of the present invention is the application of the Fbr proteins (fimbrins) as such, derivatives thereof or information derived thereof for the production of a glue or an adhesive for use in moist environments. Moist environments in this context include both aquatic environments, objects and surfaces in contact with water, sea water, fresh water, high humidity, steam and/or condensation. The applications can be found in both natural or man-made environments and even on or within an animal or human body.

Since the fimbrins to be employed in accordance with this invention are obtained from or derived from archaea cells which need extreme environmental conditions for growth, like high salt, low pH and/or high temperature, the “molecular adhesives/glues” provided in this invention are particular useful in extreme conditions, like high salt concentrations or in high temperature applications. This fact makes the herein provided uses of archaeal fimbrins as molecular glue(s) attractive in (nano)technological applications.

The present invention provides for the use of at least one protein obtained from fimbriae from archaea for the preparation of an adhesive material. As documented herein and as illustrated in the appended examples, said at least one protein is more preferably a fimbrin from archaea, and most preferably a fimbrin from M. thermoautotrophicus.

As detailed below, also a method for the preparation of an adhesive material or a glue comprising the step of isolating and/or purifying at least one protein obtained from fimbriae from archaea, namely a fimbrin, is provided in the context of this invention.

As discussed above “said at least one protein obtained from fimbriae from archaea” is preferably recombinantly produced, chemically synthesized, or chemically isolated from fimbriae. Recombinant methods for the preparation comprise, but are not limited to, amplification of the coding region (with or without the signal peptide) via PCR (introducing special restriction sites), cloning into an E. coli vector like pT7-7, or pTYB2, transformation of the resulting construct into E. coli strain ER2566 or BL21(DE3)/pLysS, expressing the protein in the recombinant strain by induction with IPTG (isopropylthio-β-D-galactoside), harvesting the cells prior to lysis, separation of cellular proteins—including the recombinant fimbrin—via SDS-PAGE, and excising the fimbrin from the gel. In the E. coli pTYB2-system introduction of the chitin-binding-domain can aid in purification of the recombinant protein; the signal peptide sequence not necessarily has to be present in the construct. Signal sequences can be predicted and/or determined by using, for example, the computer programs YinOYang or SOSUIsignal (available at http://au.expasy.org). For example, the fimbrin having the amino acid sequence shown in SEQ ID NO: 2 is believed to comprise an N-terminal 33 amino acid signal sequence.

In context of this invention, the at least one protein obtained from fimbriae from archaea is preferably a fimbrin.

Most preferably said fimbrin is a fimbrin obtained and/or derived from M. thermoautotrophicus, Methanothermobacter marburgensis, Methanobacterium formicicum, Methanobacterium bryantii, Methanothermus fervidus, Methanothermus sociabilis or is an archaeal protein obtained and/or derived from the archaea as listed in Table 1. It is also believed that the proteins derived from the bacteria as listed in Table 2 may contain an amino acid sequence which could function as a fimbrin.

In a particular preferred embodiment of the adhesive material, the use or the method of the present invention said fimbrin is encoded by a polynucleotide selected from the group consisting of
(a) a polynucleotide having a nucleotide sequence encoding the polypeptide having the deduced amino acid sequence as shown in SEQ ID NOs: 2, 4, 6;
(b) a polynucleotide having the coding sequence as shown in SEQ ID NOs: 1, 3, 5;
(c) a polynucleotide having a nucleotide sequence encoding a fragment or derivative of a polypeptide encoded by a polynucleotide of any one of (a) or (b), wherein in said derivative one or more amino acid residues are conservatively substituted compared to said polypeptide, and said fragment or derivative encodes an archaeal fimbrin;
(d) a polynucleotide having a nucleotide sequence which is at least 70% identical to a polynucleotide as defined in any one of (a) to (c) and which encodes an archaeal fimbrin;
(e) a polynucleotide having a nucleotide sequence the complementary strand of which hybridizes to a polynucleotide as defined in any one of (a) to (d) and which encodes an archaeal fimbrin; and
(f) a polynucleotide having a nucleotide sequence being degenerate to the nucleotide sequence of the polynucleotide of any one of (a) to (e);
   or the complementary strand of such a polynucleotide.

In another particular preferred embodiment of the adhesive material, the use or the method of the present invention the fimbrin is envisaged to be an archaeal protein as listed in Table 1. Also envisaged as adhesive material, in the use or method of the present invention are polynucleotides encoding said archaeal proteins as listed in Table 1, polynucleotides encoding a fragment or derivative of said archaeal proteins as listed in Table 1, polynucleotides having at least 70% identity to a polynucleotide encoding said archaeal proteins as listed in Table 1, the complementary strand of such polynucleotides encoding said archaeal proteins as listed in Table 1 as well as polynucleotides having a nucleotide sequence being degenerate to the nucleotide sequence of a polynucleotide encoding said archaeal proteins as listed in Table 1. This embodiment also pertains to the proteins as listed in Table 2 which are believed to contain an amino acid sequence which could function as a fimbrin.

The term “archaea or archaeal fimbrin” as used in context of this invention is characterized in being a functional fimbrin (or a functional fragment or derivative thereof) capable of adhering to surfaces and/or surface like structures (like grids), whereby said surfaces and surface like structure may in particular be of inorganic material, like metals, plastics and the like. Said fimbrin is derived from or naturally occurring in the “fimbriae”. It is also envisaged to cover/coat materials like carbon fibers, glass fibers, textile filaments, plastic filaments and the like with the fimbrin described herein. Also envisaged is the coating of porous material, like sponges and silica (e.g. silicium oxide) with the adhesive fimbrin protein. Also envisaged is the coating of surfaces as described herein.

The adhesive fimbrins may, accordingly, be employed to bind, stabilize and/or adhere secondary materials to primary materials. Illustratively, such a secondary material may be (without limitation) pigments, microparticles, catalyst particles, filler particles, polyelectrolyte capsules, colloidal particles, proteinaceous structures, nucleic acid molecules, and the like. Corresponding primary material may, non-limiting, be metals, silicon, alumina, silica, plastics or other oxides, polymers, fiber material (like carbon or glass fibers) and textile fibers. The adhesive fimbrins may, accordingly, also be employed to bind, stabilize and/or adhere secondary materials to primary materials, both materials being characterized mainly by a large structural difference, e.g. secondary material being a foam, non-woven, textile material or aerogel and the primary material being, non limiting, a bulk solid, sheet material or thin.

In accordance with the present invention, the term “polynucleotide” means the sequence of bases comprising purine- and pyrimidine bases which are comprised by nucleic acid molecules, whereby said bases represent the primary structure of a nucleic acid molecule. Nucleic acid sequences include DNA, cDNA, genomic DNA, RNA, synthetic forms and mixed polymers, both sense and antisense strands, or may contain non-natural or derivatized nucleotide bases, as will be readily appreciated by those skilled in the art.

When used herein, the term “polypeptide” means (a) peptide(s), (a) protein(s), or (a) polypeptide(s) which encompasses amino acid chains of a given length, wherein the amino acid residues are linked by covalent peptide bonds. However, peptidomimetics of such proteins/polypeptides wherein amino acid(s) and/or peptide bond(s) have been replaced by functional analogs are also encompassed by the invention as well as other than the 20 gene-encoded amino acids, such as e.g. selenocysteine or pyrrolysine. Peptides, oligopeptides and proteins may be termed polypeptides. The terms polypeptide and protein are often used interchangeably herein. It will be appreciated that polypeptides often contain amino acids other than the 20 amino acids commonly referred to as the 20 naturally occurring amino acids, and that many amino acids, including the terminal amino acids, may be modified in a given polypeptide, either by natural processes, such as processing and other post-translational modifications, but also by chemical modification techniques which are well known to the art. Even the common modifications that occur naturally in polypeptides are too numerous to list exhaustively here, but they are well described in basic texts and in more detailed monographs, as well as in a voluminous research literature, and they are well known to those of skill in the art.

The basic structure of polypeptides and the recombinant or synthetic production as well as isolation methods of polypeptides are well known and have been described in innumerable textbooks and other publications in the art.

The polypeptides of the present invention are shown in SEQ ID NOs.: 2, 4, 6 or in Table 1. The polypeptides listed in Table 2 may contain an amino acid sequence which could function as a fimbrin. Said polypeptides may, e.g., be a naturally purified product as described herein or a product of chemical synthetic procedures or produced by recombinant techniques from a prokaryotic or eukaryotic host (for example, by bacterial, yeast, insect, mammalian cells in culture or plant cells in culture and/or as is known in the art).

Depending upon the host employed in a recombinant production procedure, the polypeptide of the present invention may be glycosylated or may be non-glycosylated. The polypeptide of the invention may also include an initial methionine amino acid residue. The polypeptide according to the invention may be further modified to contain additional chemical moieties not normally part of the polypeptide. Those derivatized moieties may, e.g., improve the stability, solubility, the biological half life or absorption of the polypeptide. The moieties may also reduce or eliminate any undesirable side effects of the polypeptide and the like. An overview for these moieties can be found, e.g., in Remington's Pharmaceutical Sciences (18^{th} ed., Mack Publishing Co., Easton, Pa. (1990)). Polyethylene glycol (PEG) is an example for such a chemical moiety which has been used for the preparation of therapeutic polypeptides. The attachment of PEG to polypeptides has been shown to protect them against proteolysis (Sada, J. Fermentation Bioengineering 71 (1991), 137-139). Various methods are available for the attachment of certain PEG moieties to polypeptides (for review see: Abuchowski, in “Enzymes as Drugs”; Holcerberg and Roberts, eds. (1981), 367-383). Generally, PEG molecules are connected to the polypeptide via a reactive group found on the polypeptide. Amino groups, e.g. on lysines or the amino terminus of the polypeptide are convenient for this attachment among others.

The present invention also relates to the polynucleotides which encode a polypeptide, which has a homology, that is to say a sequence identity, of at least 30%, preferably of at least 40%, more preferably of at least 50%, even more preferably of at least 60% and particularly preferred of at least 70%, especially preferred of at least 80% and even more preferred of at least 90%, 95%, 96%, 97%, 98% or 99% to the amino acid sequence as shown in SEQ ID NOs.: 2, 4, 6 or to the amino acid sequence of the proteins listed in Table 1 or Table 2. Such homologs of the polypeptide of the present invention encode a fimbrin which is preferably useful as an adhesive material.

In order to determine whether a nucleic acid sequence or an amino acid sequence has a certain degree of identity to the nucleic acid sequence encoding a fimbrin or to an amino acid sequence of a fimbrin, the skilled person can use means and methods well-known in the art, e.g., alignments, either manually or by using computer programs such as those mentioned further down below in connection with the definition of the term “hybridization” and degrees of homology.

For example, BLAST2.0, which stands for Basic Local Alignment Search Tool (Altschul, Nucl. Acids Res. 25 (1997), 3389-3402; Altschul, J. Mol. Evol. 36 (1993), 290-300; Altschul, J. Mol. Biol. 215 (1990), 403-410), can be used to search for local sequence alignments. BLAST produces alignments of both nucleotide and amino acid sequences to determine sequence similarity. Because of the local nature of the alignments, BLAST is especially useful in determining exact matches or in identifying similar sequences. The fundamental unit of BLAST algorithm output is the High-scoring Segment Pair (HSP). An HSP consists of two sequence fragments of arbitrary but equal lengths whose alignment is locally maximal and for which the alignment score meets or exceeds a threshold or cutoff score set by the user. The BLAST approach is to look for HSPs between a query sequence and a database sequence, to evaluate the statistical significance of any matches found, and to report only those matches which satisfy the user-selected threshold of significance. The parameter E establishes the statistically significant threshold for reporting database sequence matches. E is interpreted as the upper bound of the expected frequency of chance occurrence of an HSP (or set of HSPs) within the context of the entire database search. Any database sequence whose match satisfies E is reported in the program output.

Analogous computer techniques using BLAST (Altschul (1997), loc. cit.; Altschul (1993), loc. cit.; Altschul (1990), loc. cit.) are used to search for identical or related molecules in nucleotide databases such as GenBank or EMBL. This analysis is much faster than multiple membrane-based hybridizations. In addition, the sensitivity of the computer search can be modified to determine whether any particular match is categorized as exact or similar. The basis of the search is the product score which is defined as:

$$\frac{\% sequence identity \times \% maximum BLAST score}{100}$$

and it takes into account both the degree of similarity between two sequences and the length of the sequence match. For example, with a product score of 40, the match will be exact within a 1-2% error; and at 70, the match will be exact. Similar molecules are usually identified by selecting those which show product scores between 15 and 40, although lower scores may identify related molecules.

The present invention also relates to nucleic acid molecules which hybridize to one of the above described nucleic acid molecules and which encode a fimbrin.

The term “hybridizes” as used in accordance with the present invention may relate to hybridization under stringent or non-stringent conditions. If not further specified, the conditions are preferably non-stringent. Said hybridization conditions may be established according to conventional protocols described, for example, in Sambrook, Russell “Molecular Cloning, A Laboratory Manual”, Cold Spring Harbor Laboratory, N.Y. (2001); Ausubel, “Current Protocols in Molecular Biology”, Green Publishing Associates and Wiley Interscience, N.Y. (1989), or Higgins and Hames (Eds.) “Nucleic acid hybridization, a practical approach” IRL Press Oxford, Washington D.C., (1985). The setting of conditions is well within the skill of the artisan and can be determined according to protocols described in the art. Thus, the detection of only specifically hybridizing sequences will usually require stringent hybridization and washing conditions such as 0.1×SSC, 0.1% SDS at 65° C. Non-stringent hybridization conditions for the detection of homologous or not exactly complementary sequences may be set at 6×SSC, 1% SDS at 65° C. As is well known, the length of the probe and the composition of the nucleic acid to be determined constitute further parameters of the hybridization conditions. Note that variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility. Hybridizing nucleic acid molecules also comprise fragments of the above described molecules. Such fragments may represent nucleic acid sequences which encode a fimbrin, and which have a length of at least 12 nucleotides, preferably at least 15, more preferably at least 18, more preferably of at least 21 nucleotides, more preferably at least 30 nucleotides, even more preferably at least 40 nucleotides and most preferably at least 60 nucleotides. Furthermore, nucleic acid molecules which hybridize with any of the aforementioned nucleic acid molecules also include complementary fragments, derivatives and allelic variants of these molecules. Additionally, a hybridization complex refers to a complex between two nucleic acid sequences by virtue of the formation of hydrogen bonds between complementary G and C bases and between complementary A and T bases; these hydrogen bonds may be further stabilized by base stacking interactions. The two complementary nucleic acid sequences hydrogen bond in an antiparallel configuration. A hybridization complex may be formed in solution (e.g., Cot or Rot analysis) or between one nucleic acid sequence present in solution and another nucleic acid sequence immobilized on a solid support (e.g., membranes, filters, chips, pins or glass slides to which, e.g., cells have been fixed). The terms complementary or complementarity refer to the natural binding of polynucleotides under permissive salt and temperature conditions by base-pairing. For example, the sequence “A-G-T” binds to the complementary sequence “T-C-A”. Complementarity between two single-stranded molecules may be “partial”, in which only some of the nucleic acids bind, or it may be complete when total complementarity exists between single-stranded molecules. The degree of complementarity between nucleic acid strands has significant effects on the efficiency and strength of hybridization between nucleic acid strands. This is of particular importance in amplification reactions, which depend upon binding between nucleic acids strands.

The term “hybridizing sequences” preferably refers to sequences which display a sequence identity of at least 40%, preferably at least 50%, more preferably at least 60%, even more preferably at least 70%, particularly preferred at least 80%, more particularly preferred at least 90%, even more particularly preferred at least 95%, 97% or 98% and most preferably at least 99% identity with a nucleic acid sequence as described above encoding a fimbrin to be employed in context of this invention, in particular as molecular glue. Moreover, the term “hybridizing sequences” refers to sequences encoding a fimbrin having a sequence identity of at least 40%, preferably at least 50%, more preferably at least 60%, even more preferably at least 70%, particularly preferred at least 80%, more particularly preferred at least 90%, even more particularly preferred at least 95%, 97% or 98% and most preferably at least 99% identity with an amino acid sequence of a fimbrin as described herein above.

In accordance with the present invention, the term “identical” or “percent identity” in the context of two or more nucleic acid or amino acid sequences, refers to two or more sequences or subsequences that are the same, or that have a specified percentage of amino acid residues or nucleotides that are the same (e.g., 60% or 65% identity, preferably, 70-95% identity, more preferably at least 95%, 97%, 98% or 99% identity), when compared and aligned for maximum correspondence over a window of comparison, or over a designated region as measured using a sequence comparison algorithm as known in the art, or by manual alignment and visual inspection. Sequences having, for example, 60% to 95% or greater sequence identity are considered to be substantially identical. Such a definition also applies to the complement of a test sequence. Preferably the described identity exists over a region that is at least about 15 to 25 amino acids or nucleotides in length, more preferably, over a region that is about 50 to 100 amino acids or nucleotides in length. Those having skill in the art will know how to determine percent identity between/among sequences using, for example, algorithms such as those based on CLUSTALW computer program (Thompson, Nucl. Acids Res. 2 (1994), 4673-4680) or FASTDB (Brutlag, Comp. App. Biosci. 6 (1990), 237-245), as known in the art.

Polynucleotides which hybridize with the polynucleotides of the invention can, in principle, encode a fimbrin or can encode modified versions thereof. Polynucleotides which hybridize with the polynucleotides disclosed in connection with the invention can for instance be isolated from genomic libraries or cDNA libraries of archaeas having a fimbrin of interest. Preferably, such polynucleotides are from archaeal origin.

The polynucleotide of the invention may also be a variant, analog or paralog of such a polynucleotide as described herein. As used herein, the term “analogs” refers to two nucleic acids that have the same or similar function, but that have evolved separately in unrelated organisms. As used herein, the term “orthologs” refers to two nucleic acids from different species, but that have evolved from a common ancestral gene by specification. Normally, orthologs encode polypeptides having the same or similar functions. As also used herein, the term “paralogs” refers to two nucleic acids that are related by duplication within a genome. Paralogs usually have different functions, but these functions may be related (Tatusov, Science 278 (1997), 631-637). Analogs, orthologs and paralogs of naturally occurring fimbrins can differ from the naturally occurring fimbrins, by post-translational modifications, by amino acid sequence differences, or by both. Post-translational modifications include in vitro chemical derivatization of polypeptides, e.g., acetylation, carboxylation, phosphorylation, or glycosylation, and such modifications may occur during polypeptide synthesis or processing or following treatment with isolated modifying enzymes. In particular, orthologs of the invention will generally exhibit at least 80-85%, more preferably, 85-90% or 90-95%, and most preferably 95%, 96%, 97%, 98% or even 99% identity or sequence identity with all or part of a naturally occurring fimbrin sequence and will exhibit a function similar to a fimbrin.

Alternatively, such polynucleotides can be prepared by genetic engineering or chemical synthesis.

Hybridizing polynucleotides may be identified and isolated by using the polynucleotides described herein/above or parts or reverse complements thereof, for instance by hybridization according to standard methods (see for instance Sambrook and Russell (2001), Molecular Cloning: A Laboratory Manual, CSH Press, Cold Spring Harbor, N.Y., USA). Polynucleotides comprising the same or substantially the same nucleotide sequence as indicated in SEQ ID NOs: 1, 3, 5 can, for instance, be used as hybridization probes. The fragments used as hybridization probes can also be synthetic fragments which are prepared by usual synthesis techniques, and the sequence of which is substantially identical with that of a polynucleotide according to the invention.

The molecules hybridizing with the polynucleotides of the invention also comprise fragments, derivatives and allelic variants of the above-described polynucleotides encoding a fimbrin. Herein, fragments are understood to mean parts of the polynucleotides which are long enough to encode the described polypeptide, preferably showing the biological activity of a polypeptide of the invention as described above. In this context, the term derivative means that the sequences of these molecules differ from the sequences of the above-described polynucleotides in one or more positions, preferably within the preferred ranges of homology mentioned above.

Preferably, the degree of homology is determined by comparing the respective sequence with the nucleotide sequence of the coding region of SEQ ID NOs: 1, 3, 5. When the sequences which are compared do not have the same length, the degree of homology preferably refers to the percentage of nucleotide residues in the shorter sequence which are identical to nucleotide residues in the longer sequence. The degree of homology can be determined conventionally using known computer programs such as the DNASTAR program with the ClustalW analysis. This program can be obtained from DNASTAR, Inc., 1228 South Park Street, Madison, Wis. 53715 or from DNASTAR, Ltd., Abacus House, West Ealing, London W13 OAS UK (support@dnastar.com) and is accessible at the server of the EMBL outstation. When using the Clustal analysis method to determine whether a particular sequence is, for instance, 80% identical to a reference sequence the settings are preferably as follows: Matrix: blosum 30; Open gap penalty: 10.0; Extend gap penalty: 0.05; Delay divergent: 40; Gap separation distance: 8 for comparisons of amino acid sequences. For nucleotide sequence comparisons, the Extend gap penalty is preferably set to 5.0.

Preferably, the degree of homology of the hybridizing polynucleotide is calculated over the complete length of its coding sequence which is described herein. It is furthermore preferred that such a hybridizing polynucleotide, and in particular the coding sequence comprised therein, has a length of at least 100 nucleotides, preferably at least 200 nucleotides, more preferably of at least 300 nucleotides, even more preferably of at least 400 nucleotides and particularly preferred of at least 500 nucleotides.

Preferably, sequences hybridizing to a polynucleotide according to the invention comprise a region of homology of at least 90%, preferably of at least 93%, more preferably of at least 95%, still more preferably of at least 98% and particularly preferred of at least 99% identity to an above-described polynucleotide, wherein this region of homology has a length of at least 100 nucleotides, preferably 200 nucleotides, more preferably of at least 300 nucleotides, even more preferably of at least 400 nucleotides and particularly preferred of at least 500 nucleotides. Homology, moreover, means that there is a functional and/or structural equivalence between the corresponding polynucleotides or polypeptides encoded thereby. Polynucleotides which are homologous to the above-described molecules and represent derivatives of these molecules are normally variations of these molecules which represent modifications having the same biological function. They may be either naturally occurring variations, or mutations, and said mutations may have formed naturally or may have been produced by deliberate mutagenesis. Furthermore, the variations may be synthetically produced sequences. The allelic variants may be naturally occurring variants or synthetically produced variants or variants produced by recombinant DNA techniques. Deviations from the above-described polynucleotides may have been produced, e.g., by deletion, substitution, insertion and/or recombination.

The polypeptides encoded by the different variants of the polynucleotides of the invention possess certain characteristics they have in common. These include for instance biological activity, molecular weight, immunological reactivity, conformation, etc., and physical properties, such as for instance the migration behavior in gel electrophoreses, chromatographic behavior, sedimentation coefficients, solubility, spectroscopic properties, stability, pH optimum, temperature optimum etc. The biological activity of a polypeptide of the invention, in particular the capacity to act as fimbrin, can be tested as is known in the art.

The invention also relates to oligonucleotides specifically hybridizing to a polynucleotide of the invention. Such oligonucleotides have a length of preferably at least 10, in particular at least 15, and particularly preferably of at least 50 nucleotides. Advantageously, their length does not exceed a length of 400, preferably 300, more preferably 200, still more preferably 100 and most preferably 50 nucleotides. The oligonucleotides of the invention can be used for instance as primers for amplification techniques such as the PCR reaction or as a hybridization probe to isolate related genes. The hybridization conditions and homology values described above in connection with the polynucleotide encoding a fimbrin may likewise apply in connection with the oligonucleotides mentioned herein.

The polynucleotides of the invention can be DNA molecules, in particular genomic DNA or cDNA. Moreover, the polynucleotides of the invention may be RNA molecules. The polynucleotides of the invention can be obtained for instance from natural sources or may be produced synthetically or by recombinant techniques, such as PCR.

In another aspect, the present invention relates to recombinant nucleic acid molecules comprising the polynucleotide of the invention described above. The term “recombinant nucleic acid molecule” refers to a nucleic acid molecule which contains in addition to a polynucleotide of the invention as described above at least one further heterologous coding or non-coding nucleotide sequence. The term “heterologous” means that said polynucleotide originates from a different species or from the same species, however, from another location in the genome than said added nucleotide sequence. The term “recombinant” implies that nucleotide sequences are combined into one nucleic acid molecule by the aid of human intervention. The recombinant nucleic acid molecule of the invention can be used alone or as part of a vector.

In a preferred embodiment, the recombinant nucleic acid molecules further comprise expression control sequences operably linked to the polynucleotide comprised by the recombinant nucleic acid molecule, more preferably these recombinant nucleic acid molecules are expression cassettes. The term “operatively linked”, as used in this context, refers to a linkage between one or more expression control sequences and the coding region in the polynucleotide to be expressed in such a way that expression is achieved under conditions compatible with the expression control sequence.

Expression comprises transcription of the heterologous DNA sequence, preferably into a translatable mRNA. Regulatory elements ensuring expression in prokaryotic as well as in eukaryotic cells are well known to those skilled in the art. They encompass promoters, enhancers, termination signals, targeting signals and the like. Examples are given further below in connection with explanations concerning vectors. In the case of eukaryotic cells, expression control sequences may comprise poly-A signals ensuring termination of transcription and stabilization of the transcript, Additional regulatory elements may include transcriptional as well as translational enhancers.

### Archaea

It can be stated, that information processing (=transcription and translation) in archaea resembles more the bacterial than the eukaryotic systems, which indicates that genetic manipulations can be done in archaea. Although, so far only some genetic markers (e.g. phenotypic markers, reporter genes) from archaea are known, it is believed that fimbrins can be expressed in archaea. Accordingly, expression of a fimbrin in its natural host under its endogenous promoter is envisaged. Expression can also be achieved by using a preferably strong constitutive or strong inducible promoter which is different from the promoter that normally controls expression of the fimbrin of interest. Alternatively, expression in a heterologous archaeal host is believed to be feasible. The term “heterologous archaeal host” means that said archaeal host is different from the archaebacterium which expresses the fimbrin of interest.

Moreover, vectors encoding the fimbrins may be used to express said proteins. These vectors are, inter alia, in particular plasmids, cosmids, viruses, YACs, BACs, bacteriophages and other vectors commonly used in genetic engineering, which contain the above-described polynucleotides of the invention. In a preferred embodiment of the invention, the vectors of the invention are suitable for the transformation of fungal cells, cells of microorganisms such as yeast or bacterial cells, animal cells or of plant cells.

The vectors may further comprise expression control sequences operably linked to said polynucleotides contained in the vectors. These expression control sequence may be suited to ensure transcription and synthesis of a translatable RNA in prokaryotic or eukaryotic cells.

The expression of the polynucleotides of the invention in prokaryotic or eukaryotic cells, for instance in Escherichia coli, is interesting because it permits a more precise characterization of the biological activities of the encoded polypeptide. Moreover, it is possible to express these polypeptides in such prokaryotic or eukaryotic cells which are free from interfering polypeptides. In addition, it is possible to insert different mutations into the polynucleotides by methods usual in molecular biology (see for instance Sambrook and Russell (2001), Molecular Cloning: A Laboratory Manual, CSH Press, Cold Spring Harbor, N.Y., USA), leading to the synthesis of polypeptides possibly having modified biological properties. In this regard it is on the one hand possible to produce deletion mutants in which polynucleotides are produced by progressive deletions from the 5′ or 3′ end of the coding DNA sequence, and said polynucleotides lead to the synthesis of correspondingly shortened polypeptides as described herein.

On the other hand, the introduction of point mutations is also conceivable at positions at which a modification of the amino acid sequence for instance influences the biological activity or the regulation of the polypeptide.

For genetic engineering in prokaryotic cells, the polynucleotides of the invention or parts of these molecules can be introduced into plasmids which permit mutagenesis or sequence modification by recombination of DNA sequences. Standard methods (see Sambrook and Russell (2001), Molecular Cloning: A Laboratory Manual, CSH Press, Cold Spring Harbor, N.Y., USA) allow base exchanges to be performed or natural or synthetic sequences to be added. DNA fragments can be connected to each other by applying adapters and linkers to the fragments. Moreover, engineering measures which provide suitable restriction sites or remove surplus DNA or restriction sites can be used. In those cases, in which insertions, deletions or substitutions are possible, in vitro mutagenesis, “primer repair”, restriction or ligation can be used. In general, a sequence analysis, restriction analysis and other methods of biochemistry and molecular biology are carried out as analysis methods.

Additionally, the present invention also describes a method for producing genetically engineered host cells comprising introducing the herein above-described polynucleotides, recombinant nucleic acid molecules or vectors encoding archaeal fimbrins into a host cell.

The useful fimbrin may be produced in host cells, in particular prokaryotic or eukaryotic cells, genetically engineered with the above-described polynucleotides, recombinant nucleic acid molecules or vectors of the invention or obtainable by the above-mentioned method for producing genetically engineered host cells, and to cells derived from such transformed cells and containing a polynucleotide, recombinant nucleic acid molecule or vector of the invention. In a preferred embodiment the host cell is genetically modified in such a way that it contains a polynucleotide stably integrated into the genome. Preferentially, the host cell of the invention is a bacterial, yeast, fungus, plant or animal cell.

More preferably the polynucleotide can be expressed so as to lead to the production of a fimbrin polypeptide. An overview of different expression systems is for instance contained in Methods in Enzymology 153 (1987), 385-516, in Bitter (Methods in Enzymology 153 (1987), 516-544) and in Sawers (Applied Microbiology and Biotechnology 46 (1996), 1-9), Billman-Jacobe (Current Opinion in Biotechnology 7 (1996), 500-4), Hockney (Trends in Biotechnology 12 (1994), 456-463), Griffiths (Methods in Molecular Biology 75 (1997), 427-440). An overview of yeast expression systems is for instance given by Hensing (Antonie van Leuwenhoek 67 (1995), 261-279), Bussineau (Developments in Biological Standardization 83 (1994), 13-19), Gellissen (Antonie van Leuwenhoek 62 (1992), 79-93), Fleer (Current Opinion in Biotechnology 3 (1992), 486-496), Vedvick (Current Opinion in Biotechnology 2 (1991), 742-745) and Buckholz (Bio/Technology 9 (1991), 1067-1072).

Expression vectors have been widely described in the literature. As a rule, they contain not only a selection marker gene and a replication-origin ensuring replication in the host selected, but also a bacterial or viral promoter, and in most cases a termination signal for transcription. Between the promoter and the termination signal there is in general at least one restriction site or a polylinker which enables the insertion of a coding DNA sequence. The DNA sequence naturally controlling the transcription of the corresponding gene can be used as the promoter sequence, if it is active in the selected host organism. However, this sequence can also be exchanged for other promoter sequences. It is possible to use promoters ensuring constitutive expression of the gene and inducible promoters which permit a deliberate control of the expression of the gene. Bacterial and viral promoter sequences possessing these properties are described in detail in the literature. Regulatory sequences for the expression in microorganisms (for instance E. coli, S. cerevisiae) are sufficiently described in the literature. Promoters permitting a particularly high expression of a downstream sequence are for instance the T7 promoter (Studier et al., Methods in Enzymology 185 (1990), 60-89), lacUV5, trp, trp-lacUV5 (DeBoer et al., in Rodriguez and Chamberlin (Eds), Promoters, Structure and Function; Praeger, New York, (1982), 462-481; DeBoer et al., Proc. Natl. Acad. Sci. USA (1983), 21-25), Ip1, rac (Boros et al., Gene 42 (1986), 97-100). Inducible promoters are preferably used for the synthesis of polypeptides. These promoters often lead to higher polypeptide yields than do constitutive promoters. In order to obtain an optimum amount of polypeptide, a two-stage process is often used. First, the host cells are cultured under optimum conditions up to a relatively high cell density. In the second step, transcription is induced depending on the type of promoter used. In this regard, a tac promoter is particularly suitable which can be induced by lactose or IPTG (=isopropyl-β-D-thiogalactopyranoside) (DeBoer et al., Proc. Natl. Acad. Sci. USA 80 (1983), 21-25). Termination signals for transcription are also described in the literature.

The transformation of the host cell with a polynucleotide or vector according to the invention can be carried out by standard methods, as for instance described in Sambrook and Russell (2001), Molecular Cloning: A Laboratory Manual, CSH Press, Cold Spring Harbor, N.Y., USA; Methods in Yeast Genetics, A Laboratory Course Manual, Cold Spring Harbor Laboratory Press, 1990. The host cell is cultured in nutrient media meeting the requirements of the particular host cell used, in particular in respect of the pH value, temperature, salt concentration, aeration, antibiotics, vitamins, trace elements etc. The polypeptide according to the present invention can be recovered and purified from recombinant cell cultures by methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Polypeptide refolding steps can be used, as necessary, in completing configuration of the polypeptide. Finally, high performance liquid chromatography (HPLC) can be employed for final purification steps.

The person skilled in the art is readily in the position to obtain a fimbrin/fimbrin protein preparation. A corresponding method may comprise the following steps

(a) culturing archaea cells with fimbriae;
(b) shearing the fimbriae from said cells;
(c) purifying said fimbriae;
(d) isolating the fimbrin from said fimbriae.

In general, culturing of archaea can be done by applying methods known in the art which may be somewhat adjusted by the skilled person to the respective archaebacterium, if deemed to be necessary. Shearing of fimbriae follows procedures known in the art which are exemplified in the appended Examples. Purifying of fimbriae can be done, for example, as described hereinabove and in the appended Examples. Isolating fimbrin from fimbriae can be, for example, done by using denaturing agents such as SDS, for example, 0.1% SDS, Triton, for example Triton X-100 and/or purification via e.g. size exclusion chromatography. Further purification techniques that may be used are, for example, anion or cation exchange chromatography, phosphocellulose chromatography, hydrophobic interaction chromatography, affinity chromatography, hydroxylapatite chromatography and the like. Polypeptide refolding steps can be used, as necessary, in completing configuration of the polypeptide. Finally, high performance liquid chromatography (HPLC) can be employed for final purification steps.

Fimbrial proteins can be purified from isolated fimbriae by the following general procedure:

Fimbriae are denatured by various treatments into the fimbrin monomers which can be purified in solutions containing those denaturing agents via e.g. chromatographic procedures, especially those separating the monomers according to their size (especially HPLC purification has to be noted here). For denaturation of fimbriae into monomers a 60 min treatment at 25° C. with a final concentration of the following detergents can be used: 0.1% SDS; or 0.05% Triton X100; or 0.05% CTAB. Solubilization of fimbriae into monomers also can be achieved by a 60 min treatment at 80° C. with a final concentration of 1.5 M guanidine hydrochloride.

As documented in the appended examples, a partial preferred fimbrin to be employed as adhesive material is obtainable from Methanothermobacter thermoautotrophicus (M. thermoautotrophicus), more preferably from M. thermoautotrophicus is M. thermoautotrophicus ΔH or Ag5 and particularly preferred from M. thermoautotrophicus ΔH as deposited under DSMZ 1053 with the “Deutsche Sammlung von Mikroorganismen und Zellkulturen”, Braunschweig. The corresponding strain was originally described in Zeikus (1972); J. Bacteriol. 109, 707-713) and reclassified by Wasserfallen et al. (2000), Int. J. Syst. Evol. Microbiol. 50, 43-53.

The fimbrin to be employed in context of this invention is preferably a fimbrin protein of 15 kDa protein, as deduced by SDS-PAGE analysis on a 12.5% gel. Corresponding methods are provided in the experimental part.

The particular preferred fimbrin is encoded by a nucleotide sequence as shown in SEQ ID NO: 1 or comprises an amino acid sequence as shown in SEQ ID NO: 2.

In a further aspect, the present application relates to a composition comprising the adhesive material as described herein or comprising at least one protein obtained or obtainable from fimbriae from archaea as described herein.

The term “composition”, as used in accordance with the present invention, relates to compositions which comprise at least one adhesive material or at least one protein obtained or obtainable from fimbriae from archaea. It may, optionally, comprise further ingredients useful as adhesive material. The composition may be in solid or liquid form and may be, inter alia, in the form of (a) powder(s), (a) solution(s) or the like. In a preferred aspect, the composition described herein is a pharmaceutical composition which may in particular be useful for the medical applications/devices as mentioned herein.

As discussed above several uses of the fimbrin provided herein are now envisaged since it was found that also in vivo the “fimbriae” of M. thermoautotrophicus are used to adhere to different surfaces. Accordingly, the present invention provides for archaeal fimbrin(s) as adhesive material/glue as described herein.

The Figures show:

FIG. 1

Comparison of bacterial flagella (S. typhimurium), archaeal flagella (P. furiosus) and archaeal fimbriae (M. thermoautotrophicus), in particular, with respect to their diameter.

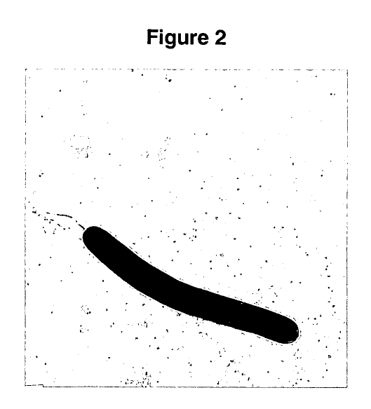

FIG. 2

TEM picture of a cell of Methanothermobacter thermoautotrophicus Ag5 grown in suspension in a serum bottle. Few cell appendages named fimbriae are visible on the cell.

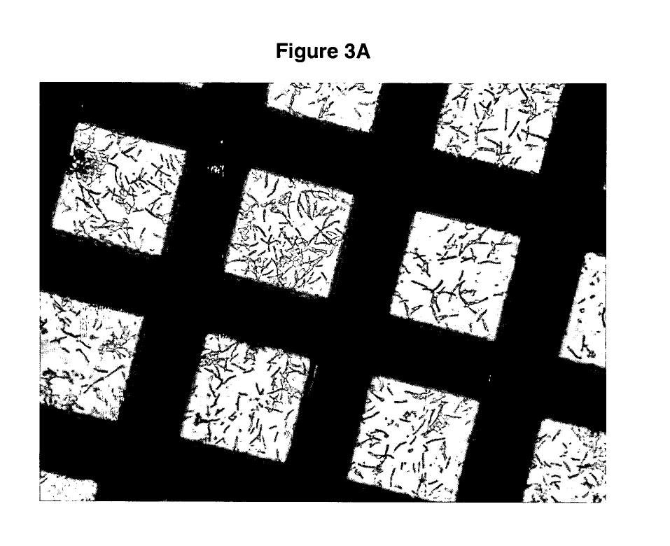

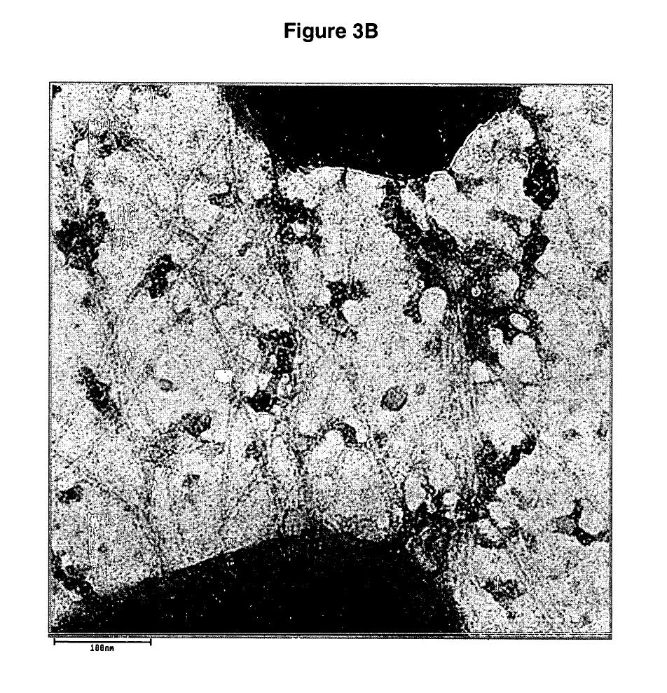

FIG. 3

TEM picture of a carbon coated gold grid incubated in a serum bottle used for growing Methanothermobacter thermoautotrophicus Ag5. Cells grow on these gold grids to a much higher density than in suspension (FIG. 3A); multiple surface appendages named fimbriae are visible on the cells (FIG. 3B).

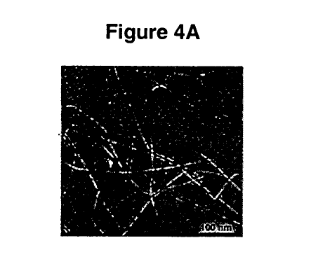

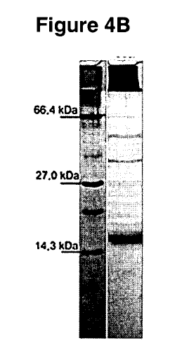

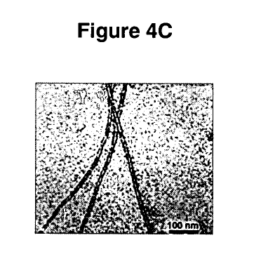

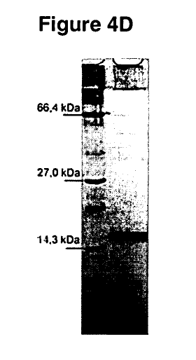

FIG. 4

Biochemical analysis of a fimbriae preparation obtained via shearing from cells and purification by isopycnic caesium chloride centrifugation.

The preparation after shearing from cells consisted mainly of fimbriae as demonstrated by TEM analysis (FIG. 4A). SDS-PAGE analysis indicated this preparation to contain one major protein of ca. 15 kDA (FIG. 4B). The band obtained after caesium chloride gradient centrifugation contained pure fimbriae as was shown by TEM analysis (FIG. 4C). This preparation resulted in one protein band migrating at 15 kDa as shown by SDS-PAGE analysis (FIG. 4D).

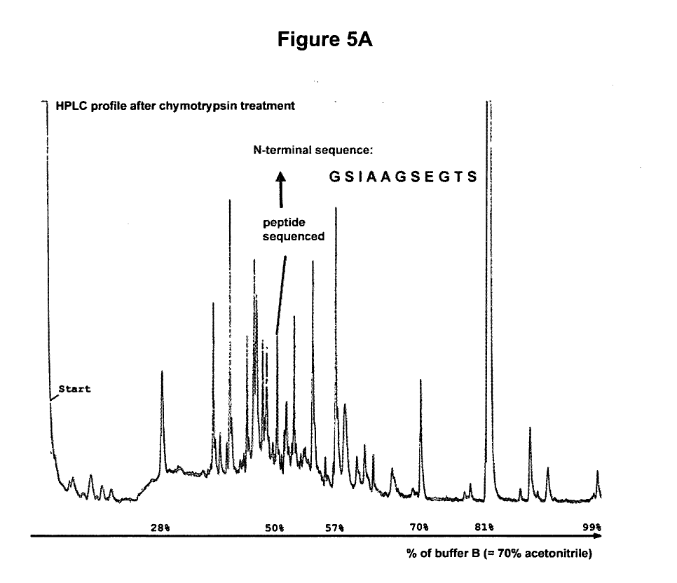

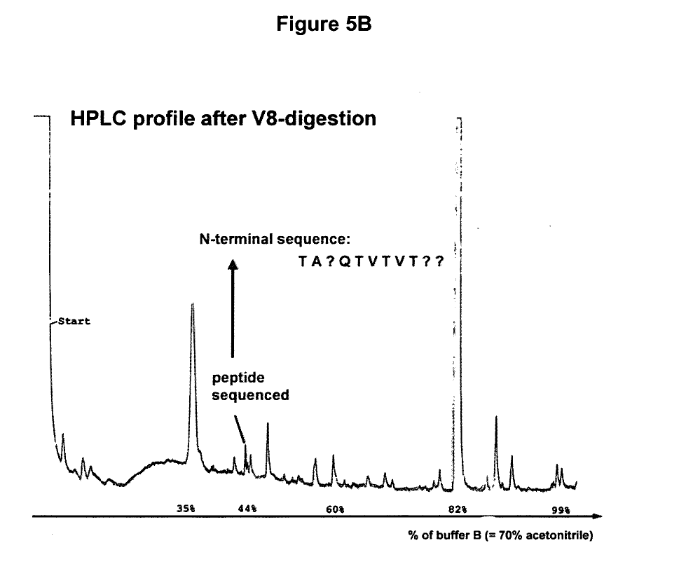

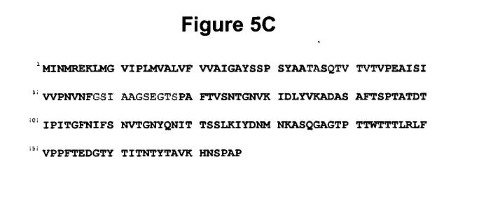

FIG. 5

Identification of M. thermoautotrophicus Fbr protein to be encoded by Mth60.

Since N-terminal sequencing of the 15 kDa protein was not possible, the denatured protein was gel-purified and the resulting protein band treated with proteases chymotrypsin and in a separate assay by V8 endoproteinase Glu-C. After extraction from the gel the resulting fragments were separated via HPLC and selected peptides were N-terminal sequenced (FIGS. 5A and 5B). The resulting sequences unambiguously identified hypothetical protein Mth60 as M. thermoautotrophicus fimbrial protein (FIG. 5C).

FIG. 6

Expression of M. thermoautotrophicus fimbrin using the IMPACT system.

The Mth60 fimbrin gene without the sequence coding for the signal peptide was amplified from genomic DNA of M. thermoautotrophicus Ag5 using PCR and primers in a way to allow cloning into pTYB2. The resulting construct was transformed into E. coli strain ER2566; induction of the fusion protein consisting of fimbrin—intein—chitin-binding-domain was via IPTG. Very clearly a fusion protein of the expected size of ca. 72 kDa is expressed only after induction. Lane S=size standard in kDa; lanes 2 to 6 give results after various times of induction (in min); lane 7 gives the result after overnight induction.

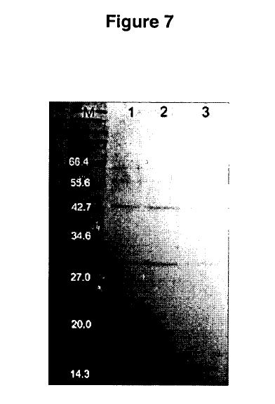

FIG. 7

Purification of recombinant M. thermoautotrophicus fimbrin.

After binding the fusion protein consisting of fimbrin—intein—chitin-binding-domain to chitin beads intein activity was induced by addition of DTT at room-temperature. The liberated fimbrin was eluted in 1 ml fractions which were analysed by SDS-PAGE. The first 3 of them are shown in lanes 1 to 3; lane M=size standard in kDa.

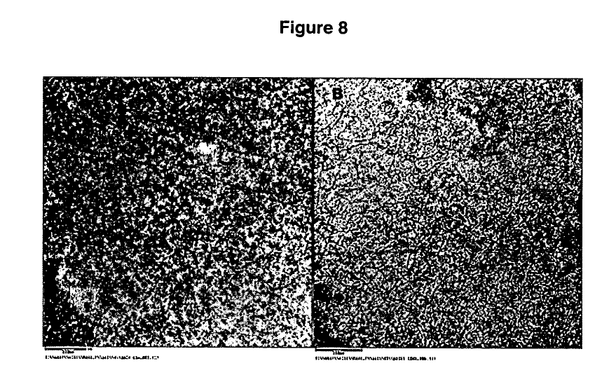

FIG. 8

In vitro self assembly of recombinant M. thermoautotrophicus fimbrin.

Purified recombinant M. thermoautotrophicus fimbrin was incubated in the presence of 20 mM CaCl₂ and 20 mM MgCl₂ (plus 2 mM NaN₃ to avoid growth of microorganisms; pH adjusted to 7.0) for 24 hours at different temperatures and analysed by TEM. No self assembly was evident if incubation was at 4° C. (A); incubation at 65° C. clearly resulted in self assembly (B). Size bars are 100 nm each.

The invention is illustrated by but not limited to the following examples.

### EXAMPLE 1

### Materials and Methods Used in this Study

### Growth of Cells and Preparation of Fimbriae

Methanothermobacter thermoautotrophicus Ag5 was cultured anaerobically in modified MS medium (Balch et al., (1979); Microbiol. Rev. 43:260-296) at 65° C. in serum bottles. Cell masses were grown anaerobically at 65° C. in a 100-liter fermentor (Bioengeneering, Wald, Switzerland) pressurized with 100 kPa of H₂/CO₂ (80:20) to stationary phase (1−2×10⁸ cells/ml; usually needing 6 days). The cell suspension was centrifuged overnight (16,500 g, Padberg Centrifuge) and the concentrate pelleted by 30 min centrifugation at 5,000 g (Juoan Centrifuge). The pellet was suspended in 100 ml aerobic MS medium, sheared with an Ultraturrax T25 (IKA-Werke, Staufen, Germany) for 1 min with 13,000 rpm and 10 s with 20,500 rpm and afterwards centrifuged twice (30 min at 17,000 g, followed by 30 min at 28,000 g; both at 4° C., Sorvall Centrifuge). This supernatant containing the fimbriae was centrifuged for 90 min at 65,000 g (70 Ti rotor, 4° C., Beckman Optima LE-80K ultracentrifuge) and the pellet containing the fimbriae resuspended in a small volume of 0.1 M HEPES-buffer (pH 7). Further purification on a CsCl-gradient (0.55 g/ml) by centrifugation for 48 h (SW60-Ti rotor at 48,000 rpm, 4° C., Beckman Optima LE-80K ultracentrifuge) resulted in one brownish band, that was isolated and dialysed exhaustively against 5 mM HEPES-dialysis-buffer (pH 7) at 4° C. The isolated fimbriae were analysed by TEM (see FIG. 4) and used for further tests. The same results are expected to be observed when culturing Methanothermobacter thermoautotrophicus ΔH (DSMZ 1053) as described herein.

### Biochemical Characterization of Fimbriae

Protein samples were resolved by electrophoresis on a 12.5% sodium dodecyl sulfate-polyacrylamide gel (SDS-PAGE—Lämmli (1970; Nature 227:680-685) and the proteins were stained with Coomassie Brilliant Blue 250 and destained with 30% methanol/10% acetic acid, or as described by Blum (1987; Electrophoresis 8:93-99) via silver staining. The method used for detection of protein glycosylation has been described by Zacharius (1969; Anal. Biochem. 30:148-152). Protein sequencing (after in gel digestion with proteinase chymotrypsin and in a separate assay by V8 endoproteinase Glu-C; see FIG. 5) was performed by the central protein analytic facility of the Biology Department of the University of Regensburg.

### Adherence Studies—Growth on Gold Grids for Transmission Electron Microscopy (TEM)

Rieger (1998; Dissertation at the University of Regensburg; Title: Elektronemikroskopische und biochemische Untersuchungen zum Aufbau des Netzwerks von Pyrodictium) has developed methods in our labs to study growth of microorganisms directly on gold grids or carbon coated gold grids. In principle gold grids are placed in small Teflon holders into serum bottles containing anaerobic medium for growing e.g. hyperthermophilic archaea. For transmission electron microscopy cells were fixed with 2.5% glutardialdehyde (final concentration) for 30 min at room temperature. In the case of cell- or fimbriae-suspensions a drop was placed for 30 s on a 200-mesh cooper grid (Plano, Wetzlar, Germany), which was covered by a carbon film. Cell suspensions were either shadowed with a Pt/C gun at 15° (DFE 50, Cressington Ltd., Wafford, UK) or negatively stained for 1 min with 2% uranyl acetate. Fimbriae in almost all cases were shadowed with a Pt/C gun at 15° (DFE 50, Cressington Ltd., Wafford, UK). Micrographs were taken with a Philips CM 12 TEM (Philips, Eindhoven, Netherlands) operating at 200 kV and a slow-scan CCD-camera (TEM 1000, TVIPS-Tietz, Gauting) with 200 ms exposure time.

### Adherence Studies—Growth on Various Other Surfaces

For these experiments various materials were added to serum bottles before autoclaving; after inoculation and growth of M. thermoautotrophicus the solids were removed and analysed for adherent cells by DAPI staining (Näther et al., (2006) J. Bacteriol. 188:6915-6923). This procedure allowed fluorescence light microscopy analyses of the not translucent materials since the UV light used for detection was provided through the objective. Detection of fimbriae via scanning electron microscopy is possible, but may sometimes be tricky. Accordingly, it is preferred to use antibodies directed against recombinant fimbrin to ask for the presence of fimbriae on adhering cells. As Table 3 shows M. thermoautotrophicus can adhere to a great variety of materials which might be of interest in nanobiotechnological applications. Notably, some of the materials tested for adherence of M. thermoautographicus may change their surface structure during autoclaving, e.g. nylon or wood, cellophane or styrofoam. Thus, if a negative result is observed when testing substrates for adherence, it does not mean that M. thermoautographicus is not able to adhere to these materials.

TABLE 3
Adherence of M. thermoautotrophicus to various surfaces

| Material | Adherence |
|---|---|
| Aluminium | ++ |
| Carbon-coated gold grids | ++ |
| Cellophane | − |
| Copper | ++ |
| Enamel | +++ |
| Glass | +++ |
| Nickel | +++ |
| Polycarbonate | + |
| PTFE | ++ |
| PVC | +++ |
| Silicium-wafer | +++ |
| Silicone | ++ |
| Steel | +++ |
| Styrofoam | − |

### EXAMPLE 2

### Structural Analysis of M. thermoautotrophicus Fimbriae

M. thermoautotrophicus Cells Possess Multiple Fimbriae

As FIG. 3B shows not only one, but more than 10 fimbriae can be observed on the surface of M. thermoautotrophicus cells, with a length of 2 to 3 μm, some are even 5 μm long. Systematic investigations indicated that their number is highest at late exponential to stationary phase.

M. thermoautotrophicus Fimbriae are Composed of Only One Fimbrin

After shearing fimbriae from cells and purification via isopycnic cesium chloride centrifugation we obtained a preparation consisting of filaments ca. 1 μm in length with a diameter of 5 nm. These filaments are composed to >95% of one protein. In FIG. 4D such a preparation was analysed via SDS-PAGE (12.5% polyacrylamide). N-terminal sequencing of the protein band at 15 kDa repeatedly was unsuccessful. Therefore the protein was in gel digested with either chymotrypsin and in a separate assay with V8 endoproteinase Glu-C; the resulting protein sequences of GSIAAGSEGTS and TA?QTVTVT?? (a “?” in the amino acid sequence obtained by protein sequencing means that the corresponding amino acid could not be identified unambiguously) unambiguously identified gene Mth60 from the published genome sequence of Methanothermobacter thermoautotrophicus strain DeltaH to code for the fimbrin. Since this constitutes the first characterization of an archaeal fimbrin comparisons to other archaeal fimbrins are not possible. Comparisons with flagellin sequences from archaea (constituting motility organelles, which are also used for adhesion) did not reveal significant homologies.

The 15 kDa protein did react specifically in a PAS (perjodat acid-Schiff) staining reaction, indicating that the fimbrin of M. thermoautotrophicus is a glycoprotein as has been reported for most archaeal flagellins (constituting another cell surface appendage of archaea, namely flagella used for motility and adhesion). No biochemical data as for specific glycosylation sites are known for any archaeal flagellins, with the exception of H. salinarum which was analysed by Wieland (1985; J. Biol. Chem. 260:15180-15185) and M. voltae which was analysed by Voisin (2005; J. Biol. Chem. 280:16586-16593).

### EXAMPLE 3

### Functional Analysis of Fimbriae

Fimbriae of M. thermoautotrophicus Enable the Cells to Adhere to Gold Grids Used for TEM

During our attempts to develop techniques to study the three-dimensional structure of M. thermoautotrophicus fimbriae (via tomography) we realized that M. thermoautotrophicus cells adhered to gold grids used for TEM (see FIG. 3A). Light microscopic studies of such gold grids (which were incubated in serum bottles used for growth) indicated that cells grew in concentrations on the gold grids which were much higher than in the liquid supernatant. Higher resolutions using TEM, clearly indicated that the cells growing on gold grids did express fimbriae to a high degree.

Existence of Fimbriae on Other Archaeal Cells and their Potential Adherence to Gold Grids Via Fimbriae

Since our data as to the adhesion of M. thermoautotrophicus to a solid surface via fimbriae was the first functional analysis of such archaeal surface appendages we tested if related archaea might possess similar fimbriae. This analysis was also done because earlier reports not necessarily did differentiate between flagella (diameter of 10 to 13 nm) and fimbriae (diameter ca. 5 nm). The data obtained can be summarized as follows:

Methanothermobacter marburgensis does possess fimbriae of ca. 5 nm diameter and adheres to gold grids (this species was newly defined in 2000 by Wasserfallen et al, Int. J. Syst. Evol. Microbiol. 50, 43-53; earlier it was listed as a subspecies of M. thermoautotrophicus).

Methanobacterium formicicum does possess fimbriae of ca. 5 nm diameter; though we could observe cells on gold grids no clear data as to the adherence of the cells via their fimbriae could be obtained.

Methanobacterium bryantii does possess fimbriae of ca. 5 nm diameter; for this species experiments as to the adherence to gold grids were not performed.

Methanothermus fervidus earlier was described to possess flagella (e.g. Jarrell et al, 1996; J. Bacteriol. 178, 5057-5064), but no pictures of these “flagella” have been published. Since only one strain of this species is available we have to state from our data that this species does not possess flagella, but rather fimbriae of ca. 5 nm diameter. The cells clearly adhere to gold grids, but only a few fimbriae could be observed on the adherent cells.

Methanothermus sociabilis is closely related to M. fervidus; again the existence of flagella has been described for this species (Boone and Castenholz, eds. Vol 1 (2001) Bergey's manual of systematic bacteriology; Springer Verlag) but again no pictures of these “flagella” are available. Our data exclude the existence of flagella on the cells, but did prove that fimbriae of ca. 5 nm diameter can be observed on those cells. A first biochemical analysis indicates that in this case the fimbrin could be a ca. 10 kDa protein.

It should be noted here that for all the above mentioned species no genome sequence data are available and therefore comparisons of the Mth60 fimbrin gene from M. thermoautotrophicus to other fimbrin genes are not possible at the moment. Nevertheless, we observed positive reactions in hybridization experiments when using, for example, the genes Mth382 and Mth383 as described herein. Initial experiments—using internal primers to amplify corresponding gene fragments directly from genomic DNA, followed by sequencing—revealed that genes coding for proteins which are homologous to the region of amino acid 68 to 98 in SEQ ID NO 4 are present, for example, in: M. marburgensis, M. bryantii, M. fervidus, and M. formicicum.

In this region only a very limited similarity between the Mth60 and Mth382/383 proteins of M. thermoautotrophicus is observed.

### EXAMPLE 4

### Cloning and Expression of M. thermoautotrophicus Fimbrin

Attempts to clone the gene Mth60 coding for the fimbrin of M. thermoautotrophicus in two different pET vectors were not successful. Also, expression in the yeast K. lactis was not possible under the conditions commonly known in the art, though the gene could be cloned into this organism. Therefore, the IMPACT system (New England Biolabs), especially pTYB2 and the Escherichia coli strain ER2566, was used for cloning and expression of a fimbrin-fusion protein. The identity of the resulting construct was verified by DNA sequencing; these data also proved that the Mth60 fimbrin gene of M. thermoautotrophicus strains DeltaH and Ag5 are identical. Our construct contains the fimbrin protein (without its signal peptide) fused at its C-terminus to an intein followed by a chitin-binding-domain. Induction of the fusion protein was found to be optimal by addition of IPTG (1 mM or preferably 0.3 mM) for 2, preferably 6 hours at 25, preferably 20° C. The intein—chitin-binding-domain should encode a protein of 57 kDa, whilst the fimbrin—intein—chitin-binding-domain fusion protein should possess a molecular mass of ca. 72 kDa. As FIG. 6 shows, a protein of the expected size clearly was expressed after induction via IPTG addition; this protein, however, was not soluble, if the conditions recommended by the supplier (New England Biolabs) were used. Yet, methods are known in the art how to solubilize this fusion protein in a way which thereafter allows its binding to chitinbeads. Addition of SH-active biochemicals like DTT or β-mercaptoethanol thereafter should induce the intein activity and thereby liberate the fimbrin.

To this end, we observed that omission of NaCl in the lysis buffer and reduction of centrifugation speeds resulted in sufficient amounts of soluble protein for further analyses. The fusion protein could be bound to chitin beads only in buffer systems without NaCl (again in contradiction to the recommended procedures), with about 50% efficiency. After addition of the SH-active chemical DTT the intein activity was best induced at room-temperature. The liberated fimbrin was eluted with 20 mM Tris/Cl (pH=7.5) in 1 ml fractions. Very interestingly bands of ca. 15 kDa, 30 kDa, 45 kDa and 60 kDa were obtained after this procedure (see FIG. 7). We assume that the higher molecular weight proteins represent multimers of the ca. 15 kDa monomer. Protein sequencing of the 15 kDa band demonstrated that it contained a mixture of the expected Mth60 protein and a small E. coli derived protein HspA. This latter protein originally was defined as a heat-shock protein and might be associated with Mth60 in a way similar to the chaperons needed for correct folding and assembly of E. coli fimbriae/pili, like type 1 pili from uropathogenic E. coli strains (M. Vetsch et al., (2004) Nature 431:329-332) or Pap pili (S. J. Hultgren and C. H. Jones; (1995) ASM News 61:457-464). Our procedure resulted in enough protein to be used in immunizations and to perform first experiments asking for self assembly. Indeed, incubations at 37° C. and 65° C., but not at 4° C. resulted in fibrillar structures of various lengths (up to 300 nm) with a diameter between 3 to 6 nm (see FIG. 8). We expect that the aforementioned protocol for expressing M. thermoautotrophicus fimbrin can also be applied for the expression of any of the fimbrins described herein which are within the scope of the present invention.

In a pilot experiment we have shown that fimbriae also can be obtained from supernatant of grown cultures of M. thermoautotrophicus. In that case cells were removed from culture medium by centrifugation at 16,000×g and the supernatant adjusted to 10.5% final concentration with polyethylene glycol (PEG 6000) plus 5.8% NaCl. After incubation for at least 12 hours at 4° C. this material was centrifuged at 11,000×g for 30 min; the resulting pellet could be further purified via CsCl centrifugation with the same procedure as outlined in example 1. First data show that this procedure results in substantial higher yields than shearing fimbriae from cells concentrated via prior centrifugation.

Accordingly, said pilot experiment can also be used for the expression of any of the fimbrins described herein which are within the scope of the present invention.

Accordingly, the present invention provides for the first evidence that the thin cell surface organelles of archaea with a diameter of ca. 5 nm, in particular of M. thermoautotrophicus enable the archaeum to adhere to different surfaces, like gold grids.

From this it is suggested that the Fbr protein(s) of archaea, in particular of M. thermoautotrophicus can be used as a molecular glue in various applications.

Fimbrin Sequences from Archaea (Fimbrin from Fimbriae)

### Methanothermobacter thermoautotrophicus Isolate DeltaH

Fimbrin MTH60-Nucleotide sequence

(SEQ ID NO: 1)

gtgatcaatatgagggaaaagttaatgggagtaatcccgcttatggttgc
ccttgtgtttgtggtggcaataggtgcatacagctcaccatcctacgcgg
caacagcaagccagacagttacagttacagtgccagaggccatctcaata
gttgtaccgaatgttaacttcgggagcattgccgcaggaagtgaaggaac
aagccctgctttcacagtaagtaacacaggtaacgtcaagatagacctct
acgtcaaggcagacgcatcagcattcacaagcccaactgctacagataca
ataccaataacagggttcaatattttcagtaatgtcacaggtaactacca
gaacatcacaaccagctcccttaagatatatgacaacatgaacaaggcat
cccagggagcaggtaccccgacaacatggacaacaacactcagactcttt
gtacccccattcacagaggacggcacatacacaataacaaacacatacac
agcagtgaagcataactcacctgcaccataa
Fimbrin MTH60-Amino acid sequence

(SEQ ID NO: 2)

MINMREKLMGVIPLMVALVFVVAIGAYSSPSYAATASQTVTVTVPEAISI
VVPNVNFGSIAAGSEGTSPAFTVSNTGNVKIDLYVKADASAFTSPTATDT
IPITGFNIFSNVTGNYQNITTSSLKIYDNMNKASQGAGTPTTWTTTLRLF
VPPFTEDGTYTITNTYTAVKHNSPAP

Methanothermobacter thermoautotrophicus Isolate DeltaH

Hypothetical fimbrin MTH382-Nucleotide sequence

(SEQ ID NO: 3)

atgtacagccacgaatggatgggtgtgctcttcatcctgctcctccttcc
ggtgccatttgcaaccatgaatacggtgcaggaagttaccgttacggtac
ccgagagtgttgaaataatggtcctctggcagggaagggaaaccgggaac
tccttcacactcacagccacagtggaacccggaaaagaatactactggcc
aggaggaccccagggactccagataaaggacctatccaacgtacccatcg
acctctacataagagccgaaggagacctccagggcccagaaaccataccc
atacagaacctcaaatacgccaactacggcatcggactccccgaaacacc
actaacaacaacctacacaccggtaagaaagaactgggcagcgaaacggg
atatcgatgcggtggtgcctgttgatctcagtctcacggtgccaccattc
acagaacccggcgaatacagggtgagggtctaccatatagccataaggtc
accgggtacctga
Hypothetical fimbrin MTH382-Amino acid sequence

(SEQ ID NO: 4)

MYSHEWMGVLFILLLLPVPFATMNTVQEVTVTVPESVEIMVLWQGRETGS
SFTLTATVEPGKEYYWPGGPQGLQIKDLSNVPIDLYIRAEGDLQGPETIP
IQNLKYANYGIGLPETPLTTTYTPVRKNWAAKRDIDAVVPVDLSLTVPPF
TEPGEYRVRVYHIAIRSPGT

Methanothermobacter thermoautotrophicus Isolate DeltaH

Hypothetical fimbrin MTH383-Nucleotide sequence

(SEQ ID NO: 5)

atgcttaaaactgctggaatggtcactgcagtcatcctccttctgctttt
aaaacctgcagcaggagccaccgcggtgcaggaggttaccgttacggtgc
ccgagagtgttgaaataatggtcctctggcagggaagggaaaccgggaac
tccttcacactcacagccacagtggaacccggaaaagaatactactggcc
aggaggaccccagggactccagataaaggacctatccaacgtacccatcg
acctctacataagagccgaaggagacctccagggcccagaaaccataccc
atacagaacctcaaatacgccaactacggcatcggactccccgaaacacc
actaacaacaacctacacaccggtaagaaagaactggatggttaaatcag
aggatgaatccctgataccggttgacctccacctcacggtaccaccagca
accgctgccggtgtctactcagttaacatataccacatagcggtccccca
tggagaatag
Hypothetical fimbrin MTH383-Amino acid sequence

(SEQ ID NO: 6)

MLKTAGMVTAVILLLLLKPAAGATAVQEVTVTVPESVEIMVLWQGRETGN
SFTLTATVEPGKEYYWPGGPQGLQIKDLSNVPIDLYIRAEGDLQGPETIP
IQNLKYANYGIGLPETPLTTTYTPVRKNWMVKSEDESLIPVDLHLTVPPA
TAAGVYSVNIYHIAVPHGE

1. An adhesive material being composed and/or consisting of at least one protein obtained or obtainable from fimbriae from archaea.

2. Use of at least one protein obtained or obtainable from fimbriae from archaea for the preparation of an adhesive material.

3. A method for the preparation of an adhesive material comprising the step of isolating and/or purifying at least one protein obtained from fimbriae from archaea.

4. The adhesive material of claim 1, the use of claim 2 or the method of claim 3, whereby said at least one protein obtained from fimbriae from archaea is recombinantly produced, chemically isolated from fimbriae or chemically synthesized.

5. The adhesive material of claim 1, the use of claim 2 or the method of claim 3, whereby said protein is a fimbrin.

6. The adhesive material, the use or the method of claim 4 or 5, whereby said fimbrin is a fimbrin obtained and/or derived from M. thermoautotrophicus, M. marburgensis, M. formicicum, M. bryantii, M. fervidus, M. sociabilis.

7. The adhesive material, the use or the method of claim of any one of claims 4 to 6, whereby said flagellin is encoded by a polynucleotide selected from the group consisting of
(a) a polynucleotide having a nucleotide sequence encoding the polypeptide having the deduced amino acid sequence as shown in SEQ ID NOs:2, 4, 6;
(b) a polynucleotide having the coding sequence as shown in SEQ ID NOs:1, 3, 5;
(c) a polynucleotide having a nucleotide sequence encoding a fragment or derivative of a polypeptide encoded by a polynucleotide of any one of (a) or (b), wherein in said derivative one or more amino acid residues are conservatively substituted compared to said polypeptide, and said fragment or derivative encodes an archaeal fimbrin;
(d) a polynucleotide having a nucleotide sequence which is at least 70% identical to a polynucleotide as defined in any one of (a) to (c) and which encodes an archaeal fimbrin;
(e) a polynucleotide having a nucleotide sequence the complementary strand of which hybridizes to a polynucleotide as defined in any one of (a) to (d) and which encodes an archaeal fimbrin; and
(f) a polynucleotide having a nucleotide sequence being degenerate to the nucleotide sequence of the polynucleotide of any one of (a) to (e);
or the complementary strand of such a polynucleotide.

8. The adhesive material, the use or the method of any one of claims 4 to 7, whereby said fimbrin is obtainable by
(a) culturing archaea cells with fimbriae;
(b) shearing the fimbriae from said cells;
(c) purifying said fimbriae;
(d) isolating the fimbrin from said fimbriae by using denaturing agents

9. The adhesive material, the use or the method of any one of claims 4 to 8, whereby said fimbrin is obtainable from Methanothermobacter thermoautotrophicus (M. thermoautotrophicus).

10. The adhesive material, the use or the method of claim 9, whereby said M. thermoautotrophicus is M. thermoautotrophicus ΔH (DSMZ 1053) or M. thermoautotrophicus Ag5.

11. The adhesive material, the use or the method of claim 9 or 10, whereby said fimbrin is a 15 kDa protein.

12. The adhesive material, the use or the method of any one of claims 9 to 11, whereby said fimbrin is encoded by a nucleotide sequence as shown in SEQ ID NO: 1 or wherein said fimbrin is or comprises an amino acid sequence as shown in SEQ ID NO: 2.

13. A composition comprising the adhesive material of any one of claims 1 or 3 to 12 or at least one protein as defined in claim 2.

14. The composition of claim 13 which is a pharmaceutical composition.
